(19) United States
(12) Patent Application Publication
(10) Pub. No.: US 2005/0113700 A1
(43) Pub. Date: May 26, 2005

(54) Ultrasonic probe

(76) Inventors: Koji Yanagihara, Tokyo (JP); Tadashi Tsukahara, Tokyo (JP); Mitsuhiro Nozaki, Tokyo (JP); Timothy Maack, Tempe, AZ (US)

Correspondence Address: Patrick W. Rasche;Armstrong Teasdale LLP, Suite 2600, One Metropolitan Square, St. Louis, MO, 63102, US

(21) Appl. No.: 10/723,767

(22) Filed: Nov. 26, 2003

(51) Int. Cl. A61B 8/00

(52) U.S. Cl. 600/459

(57) ABSTRACT

The present invention provides an ultrasonic probe, which is soft for the subject, and which has an ultrasonic transceiver unit and an enclosure for housing the unit. The integrated enclosure includes a first partial enclosure made of hard plastics and having an opening at one end, and a second partial enclosure molded together with the first partial enclosure so as to cover the opening to expand beyond the end, the transmission/reception surface of the ultrasonic transceiver unit being in contact therewith from inside.

### BACKGROUND OF THE INVENTION

The present invention relates to an ultrasonic probe and more particularly to an ultrasonic probe including an ultrasonic transceiver unit and an enclosure for housing the unit.

The ultrasonic probe is used for transmitting and receiving ultrasonic waves by contacting to a patient, for ultrasonic diagnosis. The ultrasonic probe houses a transceiver unit such as an ultrasonic transducer array in an enclosure made of plastics. The enclosure has an opening at a tip for transmitting and receiving ultrasonic waves, from which opening the transmission/reception surface of the transceiver unit is exposed (see for example the patent reference 1 below).
JP-A-2003-164450 (pp. 8-9, FIG. 13-14)

An ultrasonic probe having a structure as described above includes, at the end surface of transmitting and receiving ultrasonic waves, a joint between the transceiver unit and the enclosure, and the enclosure side is made of hard plastics with the joint being as a boundary. The hard plastics are pressed to the patient's body, forcibly imposing a burden to the patient.

### BRIEF DESCRIPTION OF THE INVENTION

An object of the present invention is to achieve an ultrasonic probe, which is soft to the subject.

The present invention has been made in view of the above circumstances and has an object to overcome the above problem and to provide an ultrasonic probe having an ultrasonic transceiver unit and an enclosure for housing the unit, the enclosure including: a first partial enclosure made of hard plastics having an opening at the tip; and a second partial enclosure integrally formed with the first partial enclosure so as to cover the opening to extend from the tip, the second partial enclosure being made of soft plastics and having a transmission/reception surface of the ultrasonic transceiver unit in contact therewith from inside.

It is preferable that the integrated molding of the first partial enclosure and the second partial enclosure is done by double molding for the purpose of effective molding. Also it is preferable that the part of the second partial enclosure in contact with the transmission/reception surface is a thin film, for decreasing the attenuation of ultrasonic waves transmitted therethrough.

The hard plastics may be preferably one of thermoplastic resins including polycarbonate, poly-butylene-terephthalate, and ABS resin, for obtaining an appropriate strength. The soft plastics may be preferably a thermoplastic polymer for obtaining an appropriate softness.

Preferably, the ultrasonic transceiver unit has an ultrasonic transducer array for the beam forming of ultrasonic waves by a phased array. Preferably, the ultrasonic transducer array has an acoustic lens on the transmission/reception surface for the convergence of ultrasonic beam. In addition the second partial enclosure may have preferably a color corresponding to the center frequency of ultrasonic waves for facilitating distinguishing of the center frequency.

In the present invention the enclosure includes a first partial enclosure made of hard plastics having an opening at the tip, and a second partial enclosure made of soft plastics integrally molded with the first partial enclosure so as to cover the opening to extend from the tip, and having a transmission/reception surface of the ultrasonic transceiver unit in contact therewith from inside. For use, the second partial enclosure made of soft plastics is pressed to the patient's body. This may soften the touch to the subject.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram of an ultrasonic diagnosing apparatus.

FIG. 2 is a schematic diagram of appearance of an ultrasonic probe.

FIG. 3 is a schematic diagram of a section in the proximity of transmission/reception end of an ultrasonic probe.

FIG. 4 is a partly enlarged schematic diagram of a section in the proximity of transmission/reception end of an ultrasonic probe.

FIG. 5 is a schematic diagram of an ultrasonic transducer array.

FIG. 6 is a schematic diagram of colorings of the transmission/reception end of an ultrasonic probe.

### DETAILED DESCRIPTION OF THE INVENTION

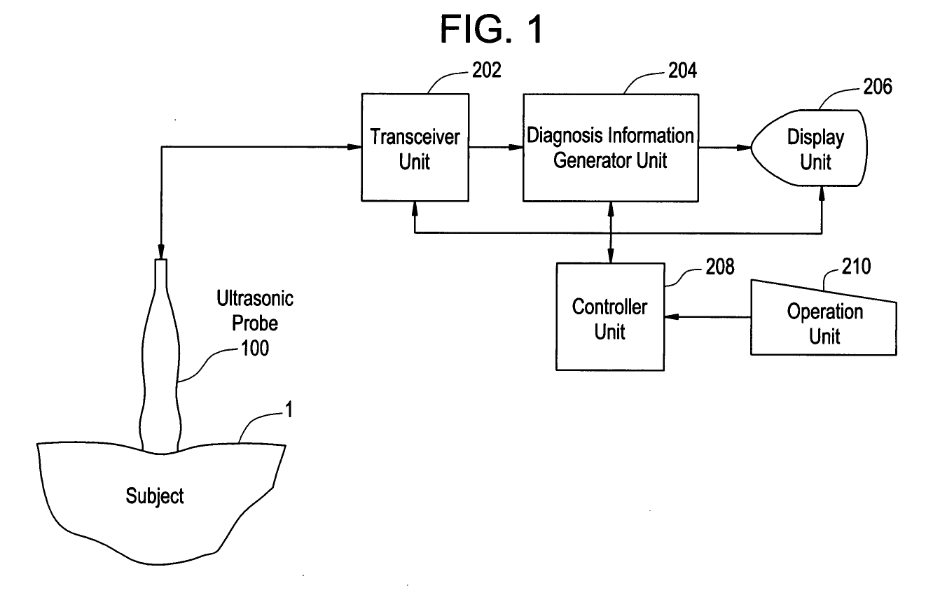

A detailed description of one preferred embodiment embodying the present invention will now be given referring to the accompanying drawings. Now referring to FIG. 1, there is shown a schematic block diagram of an ultrasonic diagnosing apparatus. As shown in the figure, the ultrasonic diagnosing apparatus includes an ultrasonic probe 100. The ultrasonic probe 100 is used so as to press onto a subject 1. The ultrasonic probe 100 is an exemplary best mode for carrying out the ultrasonic probe according to the present invention. This arrangement illustrates a preferred embodiment of the ultrasonic probe in accordance with the present invention.

The ultrasonic probe 100 is connected to a transceiver unit 202. The transceiver unit 202 gives the ultrasonic probe 100 driving signals to transmit ultrasonic waves. The transceiver unit 202 also receives echo signals received by the ultrasonic probe 100.

The transceiver unit 202 is connected to a diagnosis information generation unit 204. The diagnosis information generation unit 204 is input with received echo signals through the transceiver unit 202 and generates diagnosis information based on thus received echo signals.

For the diagnosis information, for example, an image such as a B-mode image, a color Doppler image, a Doppler spectrum image is generated. The B-mode image displays a tomography image of the subject to be diagnosed. The color Doppler image displays a velocity distribution image such as blood flow in the subject to be diagnosed. The Doppler spectrum image displays the spectra of the Doppler signals. The diagnosis information as above may be displayed on a display unit 206 connected to the diagnosis information generation unit 204.

The transceiver unit 202, diagnosis information generation unit 204 and display unit 206 are all controlled by a controller unit 208. The controller unit 208 is connected to an operation unit 210. The operation unit 210 is operated by an operator-user, in order to input appropriately commands and information given to the controller unit 208.

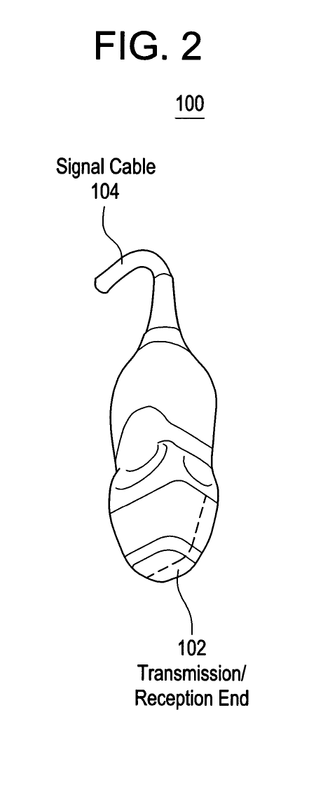

Now referring to FIG. 2, there is shown an appearance of an ultrasonic probe 100. As shown in the figure, the ultrasonic probe 100 has an approximately cylindrical outer shape. The tip of this cylindrical configuration is a transmission/reception end 102. At the other end opposed to the transmission/reception end 102 the signal cable 104 is mounted in order to connect to an ultrasonic diagnosing apparatus body.

The outer surface of the ultrasonic probe 100 is configured as an enclosure integrally formed of such a material as plastics. In the enclosure, an ultrasonic transceiver unit comprised mainly of the ultrasonic transducer is housed.

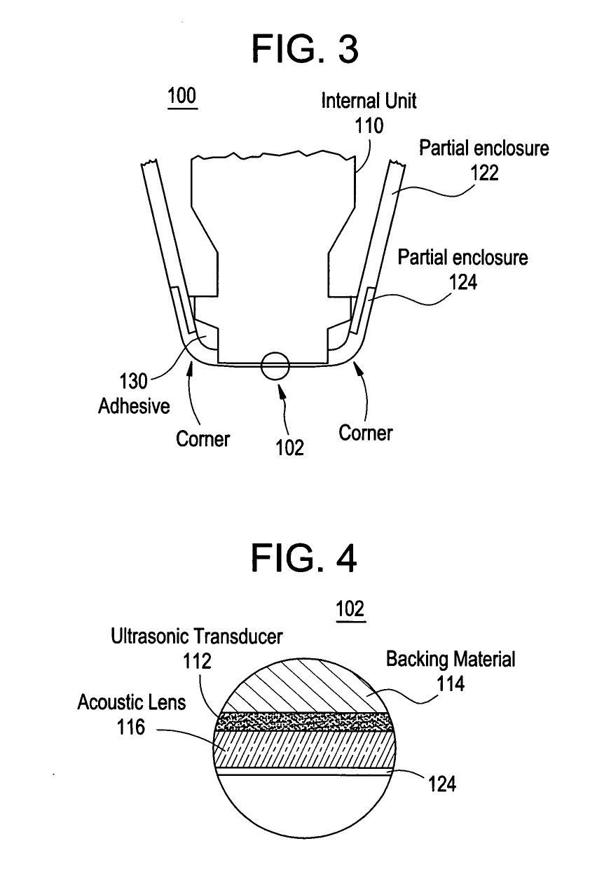

Now referring to FIG. 3, there is shown a sectional view of the transmission/reception end 102. This cross-section is taken along the dotted line shown in FIG. 2. As shown in the figure, the tip of internal unit 110 abuts to the inside of transmission/reception end 102. The enclosure in the vicinity of the transmission/reception end 102 is integrally composed of two partial enclosures 122 and 124.

The internal unit 110 is an exemplary embodiment of an ultrasonic transceiver unit in accordance with the present invention. The partial enclosure 122 is an exemplary embodiment of the first partial enclosure in accordance with the present invention. The partial enclosure 124 is an exemplary embodiment of the second partial enclosure in accordance with the present invention.

The partial enclosure 122 has an approximately cylindrical shape with an opening at one end, which is the main body of the enclosure of the ultrasonic probe 100. The partial enclosure 122 is formed of hard plastics such as polycarbonate. The polycarbonate is preferable as a material, which has a sufficient strength against an external force applied thereto during use. The partial enclosure 122 may be formed from any appropriate hard plastics materials other than polycarbonate. Some examples of such plastics materials include thermal-plastics resin such as, for example, poly-butylene-terephthalate and ABS resin.

The partial enclosure 124 is integrally formed so as to cover the opening at the end of the partial enclosure 122. The partial enclosure 124 serves as a cap with respect to the opening of the partial enclosure 122, and extends forwardly from the tip of the partial enclosure 122.

A tip end of the internal unit 110 abuts against the partial enclosure 124 from the inside. The internal unit 110 and partial enclosures 122 and 124 are adhered by means of an adhesive 130. The partial enclosure 124 has a part in contact with the tip end of the internal unit 110 formed as a thin film, and other parts formed with an appropriate wall thickness sufficient for holding the shape by itself.

The partial enclosure 124 is formed of a soft plastics material such as a thermoplastic polymer. The thermoplastic polymer is preferable because of its appropriate softness. The partial enclosure 124 may also be formed of any other soft plastics material than the thermoplastic polymer.

The enclosure formed by the partial enclosures 122 and 124 are integrally formed by double molding. Double molding is a technique well known in the art of plastic mold engineering, commonly used for the molding integrated from a plurality of plastics materials that each has a different characteristics.

Since the partial enclosure 124 is integrated to the partial enclosure 122 in such a form that it projects beyond the tip end of the partial enclosure 122, only the partial enclosure 124 is in contact with the patient's body when using.

The material for the partial enclosure 124 is made of soft plastics, which applies to the body very softly, allowing a considerable decrease of the burden of the patient, in comparison with the conventional enclosure of the ultrasonic probes made of hard plastics which contacts the patient's body. In addition, the corners as shown by the arrow on the partial enclosure 124 may be designed to be an arc of relatively large curvature, which also contributes to the decrease of burden of the patient.

Now referring to FIG. 4, there is shown an enlarged schematic view of the part of the internal unit 110 abutting to the partial enclosure 124. This figure corresponds to the circle shown in FIG. 3. As shown in the figure, the internal unit 110 has an ultrasonic transducer 112. The ultrasonic transducer 112 includes a backing filler 114 on the backside, and an acoustic lens 116 at the front face. The acoustic lens 116 is used for converging the ultrasonic beam. There is an acoustic matching layer between the ultrasonic transducer 112 and the acoustic lens 116. The front face of the acoustic lens 116 is in contact with the backside of the thin film part of the partial enclosure 124. As the partial enclosure 124 is thin film in this part, the attenuation of ultrasonic waves can be negligible in this part.

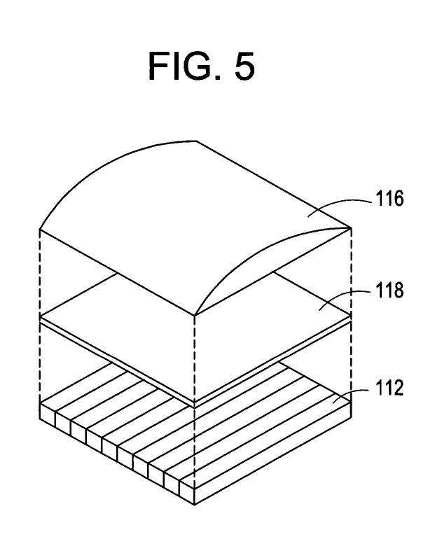

The ultrasonic transducer 112 forms an array, which is made of a plurality of transducers as shown in FIG. 5, on which the acoustic lens 116 is bonded with an acoustic matching layer 118 sandwiched therebetween. Since the ultrasonic transducer 112 forms an array, beam forming and beam steering of ultrasound waves may be achievable by applying the phased array technology.

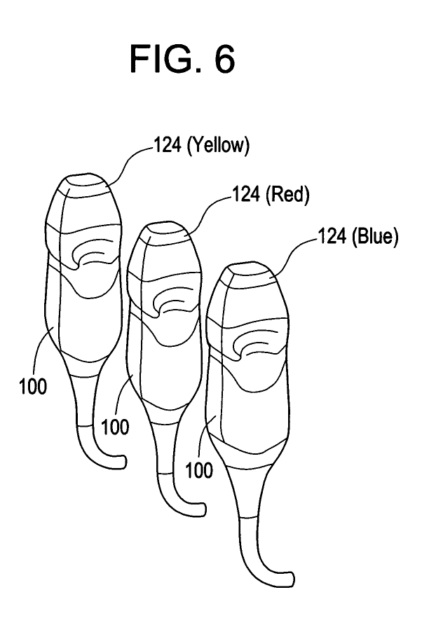

The partial enclosure 124 may have, as shown in FIG. 6, some colorings. This allows facilitating the identification of ultrasonic center frequency of the probe. In other words, yellow indicates a center frequency of 2 MHz, red indicates 5 MHz, and blue indicates 10 MHz, and so on.

The frequency indication by coloring system may be complied with the numerical representation by the color system of resistors. That is, a color and its corresponding number may be as follows: brown=1, red=2, orange=3, yellow=4, green=5, blue=6, purple=7, gray=8, and white=9.

The ultrasonic probe 100 is held, when hanging up in the probe holder of an ultrasonic diagnosing apparatus, with the transmission/reception end 102 up and the signal cable down. Since the partial enclosure 124 as the transmission/reception end 102 has different colors for center frequency, the operator—user may identify the center frequency of that specific probe at a glance.

Many widely different embodiments of the invention may be configured without departing from the spirit and the scope of the present invention. It should be understood that the present invention is not limited to the specific embodiments described in the specification, except as defined in the appended claims.

While the invention has been described in terms of various specific embodiments, those skilled in the art will recognize that the invention can be practiced with modification within the spirit and scope of the claims.

1. An ultrasonic probe having an ultrasonic transceiver unit and an enclosure for housing the unit, comprising:
a first partial enclosure formed of hard plastics having an opening at the tip; and
a second partial enclosure integrally formed with the first partial enclosure so as to cover the opening to extend from the tip, the second partial enclosure being formed of soft plastics and having the transmission/reception surface of the ultrasonic transceiver unit in contact therewith from inside.

2. An ultrasonic probe according to claim 1, wherein the integrated molding of the first partial enclosure and the second partial enclosure is performed by double molding.

3. An ultrasonic probe according to claim 1, wherein the part of the second partial enclosure in contact with the transmission/reception surface is a thin film.

4. An ultrasonic probe according to claim 1, wherein the hard plastics is polycarbonate.

5. An ultrasonic probe according to claim 1, wherein the hard plastics is poly-butylene-terephthalate.

6. An ultrasonic probe according to claim 1, wherein the hard plastics are ABS resin.

7. An ultrasonic probe according to claim 1, wherein the hard plastics are thermoplastic resin.

8. An ultrasonic probe according to claim 1, wherein the soft plastics are thermoplastic polymer.

9. An ultrasonic probe according to claim 1, wherein the ultrasonic transceiver unit includes an ultrasonic transducer array.

10. An ultrasonic probe according to claim 9, wherein the ultrasonic transducer array include an acoustic lens on the transmission/reception surface.

11. An ultrasonic probe according to claim 1, wherein the second partial enclosure has a color corresponding to the center frequency of ultrasonic waves.
